(19) United States
(12) Patent Application Publication
(10) Pub. No.: US 2023/0277831 A1
(43) Pub. Date: Sep. 7, 2023

(54) Focused Ultrasound Device

(71) Applicant: Washington University, St. Louis, MO (US)

(72) Inventors: Hong CHEN, St. Louis, MO (US); Zhongtao HU, St. Louis, MO (US); Si CHEN, St. Louis, MO (US); Yaoheng YANG, St. Louis, MO (US); Yan GONG, St. Louis, MO (US)

(73) Assignee: Washington University, St. Louis, MO (US)

(21) Appl. No.: 18/179,966

(22) Filed: Mar. 7, 2023

Related U.S. Application Data
(60) Provisional application No. 63/317,387, filed on Mar. 7, 2022.

(51) Int. Cl. A61M 37/00

(52) U.S. Cl. CPC A61M 37/0092; A61M 2210/0693; A61M 2209/088

(57) ABSTRACT

Devices, systems, and methods for performing focused ultrasound-mediated blood-brain barrier opening (FUS-BBBO) are disclosed. Focused ultrasound (FUS) devices are disclosed that include an adapter configured to attach to an actuated mounting shaft of a stereotaxic system, a transducer housing configured to mount to the adapter, and a FUS transducer housed within the transducer housing. A FUS system is disclosed that includes the disclosed FUS device operatively coupled to a transducer driving system. A stereotaxic-guided FUS-BBB system is disclosed that includes the disclosed FUS system mounted to an actuated mounting shaft of a stereotaxic system.

### CROSS-REFERENCE TO RELATED APPLICATIONS

This application claims priority from U.S. Provisional Application Ser. No. 63/317,387 filed on Mar. 7, 2022, which is incorporated herein by reference in its entirety.

### STATEMENT REGARDING FEDERALLY SPONSORED RESEARCH OR DEVELOPMENT

This invention was made with government support under EB027223, EB030102, and MH116981 awarded by the National Institutes of Health and under N00014-19-1-2335 awarded by the Office of Naval Research. The government has certain rights in the invention.

### MATERIAL INCORPORATED-BY-REFERENCE

Not applicable.

### FIELD OF THE INVENTION

The present disclosure generally relates to focused ultrasound devices for use in microbubble-mediated blood-brain barrier (BBB) opening (FUS-BBBO).

### BACKGROUND OF THE INVENTION

Focused ultrasound combined with microbubble-mediated blood-brain barrier opening (FUS-BBBO) has been established as a promising technique for the noninvasive and localized delivery of various therapeutic agents to the brain. Its feasibility and safety have been demonstrated in patients with various brain diseases, including brain tumors, Parkinson's disease, amyotrophic lateral sclerosis, and Alzheimer's disease. However, FUS-BBBO is not only a promising technique for clinical applications but also a powerful preclinical research tool that has the potential to be adopted by a broad research community, including but not limited to neuroscience, neuro-oncology, and neurology. For example, FUS-BBBO can facilitate the delivery of gene vectors encoding channelrhodopsin to the mouse brains for optogenetic neuromodulation and engineered G-protein-coupled receptors for chemogenetic neuromodulation. It can also modulate brain function by delivering neurotransmitters (e.g., GABA) to a targeted brain location. Besides these applications in neuroscience, FUS-BBBO has been used in neuro-oncology research to evaluate the delivery efficiency and therapeutic efficacy of various agents in murine models of brain tumors, such as chemotherapeutic agents (e.g., BCNU), monoclonal antibodies (e.g., Herceptin and bevacizumab), and nanoparticles (e.g., brain-penetrating nanoparticles and radiolabeled copper nanoclusters). FUS-BBBO without any drugs was found to reduce the amyloid plaque and improve cognitive performance in Alzheimer's disease mouse models. FUS-BBBO can also be used to deliver therapeutic agents (e.g., GSK-3 inhibitor) to further reduce plaque deposition in mouse models. Despite the great promise, the adoption of FUS-BBBO by the broad research community is limited by the lack of affordable, easy-to-use, and high-precision FUS devices for mouse studies.

Existing FUS devices for FUS-BBBO in mice are expensive, bulky, and with a high technical barrier. There are only a few commercially available FUS devices for preclinical FUS-BBBO research, for example, magnetic resonance imaging (MRI)-guided FUS devices provided by Image Guided Therapy (IGT, Pessac, France), MRI- and stereotactic-guided FUS devices from FUS Instruments Inc. (Toronto, Ontario, Canada), and VIFU 2000 from Alpinion US Inc. (Bothell, Wash., USA). These commercial devices are expensive (˜$50,000 to ˜$250,000). There are also custom-made FUS devices, but the cost of those systems is within the same range. Single-element FUS transducers have been the most widely used devices because they are relatively more affordable than phased arrays. However, even single-element FUS transducers are often bulky with large apertures (˜50 mm). These bulky transducers require heavy 3D motors to control their positioning for brain targeting. Moreover, MRI or ultrasound imaging is often needed to guide the spatial targeting of the FUS transducer at a specific brain location, limiting the usage of FUS-BBBO to mainly groups with expertise in ultrasound and/or MRI. A stereotactic-guided FUS system was introduced that uses a stereotactic frame to stabilize the mouse head and use the brain atlas to guide the positioning of the FUS transducer, which avoids the need for MRI or ultrasound imaging guidance. However, same as all other FUS devices, the FUS transducer is bulky. The cost of the device is still high (˜$50,000).

The broad application of FUS-BBBO in small animal research is also limited by the low spatial precision of existing FUS devices. The commonly used FUS transducers have low frequencies 1.5 MHz) with a focal region at the scale of 1×1×10 mm³, which essentially covers the entire depth of the mouse brain (˜6 mm). Low-frequency transducers are needed to minimize skull-induced attenuation and beam aberration, which is critical in clinical applications. However, the mouse skull is much thinner than the human skull. Successful BBBO was reported using a diagnostic ultrasound imaging probe with a center frequency of 8 MHz in combination with microbubbles. However, the focal region size of the ultrasound imaging probe was large, which was not suitable for spatially precise BBBO in mice. FUS transducers with optimized design are needed to precisely target individual structures in the mouse brain.

Precise control of BBBO volume and drug delivery efficiency is also needed to ensure the robust application of FUS-BBBO. Both mechanical index (defined by the ratio between acoustic pressure and the squared root of frequency, MI=P/\sqrt{f}) and cavitation index (defined by the ratio between acoustic pressure and the squared root of frequency, MI=P/\sqrt{f}) have been proposed to evaluate the likelihood of FUS-BBBO as well as the drug delivery efficiency. McDannold et al. found that MI was correlated with the threshold of FUS-induced BBB opening. Chu et al. found both MI and CI were highly correlated with the delivery efficiency of FUS-BBBO, and the correlation with MI was slightly higher than that of CI.

### SUMMARY OF THE INVENTION

Among the various aspects of the present disclosure is the provision of a focused ultrasound device.

Briefly, therefore, the present disclosure is directed toward a focused ultrasound device and uses thereof.

The present teachings include a description of a focused ultrasound (FUS) device. In one aspect, the device is configured to deliver FUS to a targeted tissue. The FUS device includes an adapter configured to an actuated mounting shaft of a stereotaxic system. The adapter includes an adapter platform. The adapter platform defines a mounting bore at one end, where the mounting bore is configured to receive the actuated mounting shaft. The adapter further includes a transducer housing bore configured to receive a transducer housing. The FUS device further includes a focused ultrasound (FUS) transducer. The FUS device further includes the transducer housing. The transducer housing includes a transducer bore connecting a transducer receptacle and a coupling cone positioned on opposite faces of the transducer housing. The transducer housing further includes the transducer receptacle configured to receive and house the focused ultrasound (FUS) transducer. The transducer housing further includes a coupling cone projecting downward from the transducer bore that is configured to insert through the transducer housing bore. In some aspects, the adapter further includes a first pair of magnets attached to the adapter within a corresponding pair of magnet insets formed within the adapter on either side of the transducer housing bore. In some aspects, the transducer housing further includes a second pair of magnets attached to the transducer housing within a corresponding second pair of magnet insets formed within the transducer housing on either side of the transducer bore. In some aspects, the first and second sets of magnets are configured to lock the transducer housing to the adapter when the coupling cone is inserted through the transducer housing bore. In some aspects, the coupling cone is further configured to contain an impedance-matched ultrasound coupling material, where the impedance-matched ultrasound coupling material is configured to provide a low-impedance acoustic path for delivery of FUS to the targeted tissue. In some aspects, the FUS device further includes a pointer insert configured to insert into the transducer mounting bore and to provide a visual indication of the position and orientation of the FUS produced by the device. In some aspects, the pointer insert includes a flanged insert configured to insert into the transducer mounting bore and a shaft extending downward and ending in a pointer tip, wherein the pointer tip is positioned at the focus point of the focused ultrasound produced by the device.

The present teachings include a description of a FUS system. In one aspect, the FUS system includes an adapter configured to couple to an actuated mounting shaft of a stereotaxic system. The adapter includes an adapter platform. The adapter platform includes a mounting bore at one end, where the mounting bore is configured to receive the actuated mounting shaft. The adapter platform further includes a transducer housing bore configured to receive a transducer housing. The FUS system further includes a focused ultrasound (FUS) transducer and at least one transducer housing. Each transducer housing includes a transducer bore connecting a transducer receptacle and a coupling cone positioned on opposite faces of the transducer housing, as well as a transducer receptacle configured to receive and house the focused ultrasound (FUS) transducer. The transducer house further includes a coupling cone projecting downward from the transducer bore, where the coupling cone is configured to insert through the transducer housing bore. The FUS system further includes a transducer driving system operatively coupled to focused ultrasound (FUS) transducer. The transducer driving system includes a function generator configured to control the operation of the FUS transducer to produce FUS, and a power amplifier configured to control the operation of the FUS transducer to produce FUS.

The present teachings also include a description of a stereotaxic-guided focused ultrasound-blood brain barrier (FUS-BBB) system. The FUS-BBB system includes a FUS system that includes an adapter configured to couple to an actuated mounting shaft of a stereotaxic system, a FUS transducer, and at least one transducer housing. In another aspect, the FUS-BBB system can include a stereotaxic system that can include an actuated mounting shaft, wherein the actuated mounting shaft is inserted into a mounting bore defined within the platform of the FUS device to couple the FUS device to the stereotaxic system. The adapter includes an adapter platform defining a mounting bore at one end, where the mounting bore is configured to receive the actuated mounting shaft as well as a transducer housing bore configured to receive at least one transducer housing. Each transducer housing includes a transducer bore connecting a transducer receptacle and a coupling cone positioned on opposite faces of the transducer housing, where the transducer receptacle is configured to receive and house the focused ultrasound (FUS) transducer. The transducer housing further includes a coupling cone projecting downward from the transducer bore, where the coupling cone is configured to insert through the transducer housing bore. The stereotaxic system include the actuated mounting shaft, wherein the actuated mounting shaft is inserted into the mounting bore defined within the platform of the FUS device to couple the FUS device to the stereotaxic system.

### DESCRIPTION OF THE DRAWINGS

The patent or application file contains at least one drawing executed in color. Copies of this patent or patent application publication with color drawing(s) will be provided by the Office upon request and payment of the necessary fee.

Those of skill in the art will understand that the drawings, described below, are for illustrative purposes only. The drawings are not intended to limit the scope of the present teachings in any way.

FIG. 1 is a drawing illustrating the elements of a focused ultrasound system in accordance with one aspect of the invention.

FIG. 2A is a drawing illustrating a top view of an adapter.

FIG. 2B is a drawing illustrating a bottom view of the adapter of FIG. 2A.

FIG. 3A is a drawing illustrating a top view of a transducer housing.

FIG. 3B is a drawing illustrating a bottom view of the transducer housing of FIG. 3A.

FIG. 4 is an image of a focused ultrasound (FUS) transducer mounted in a transducer housing.

FIG. 5 is an image of the focused ultrasound (FUS) transducer and transducer housing of FIG. 4 mounted in an adapter.

FIG. 6A is a drawing illustrating a top view of a pointer.

FIG. 6B is a drawing illustrating a bottom view of the pointer of FIG. 6A.

FIG. 7 is an image of the focused ultrasound (FUS) transducer and transducer housing of FIG. 4 mounted in the adapter of FIG. 1.

FIG. 8A is a picture of the stereotactic-guided FUS system.

FIG. 8B is a picture of FUS transducers with frequencies of 1.5, 3.0, and 6.0 MHz.

FIG. 8C is a picture of brain targeting in a mouse with the FUS system: (top) a 3D-printed pointer was aligned with the bregma in the mouse skull that was visible through the scalp, (bottom) the pointer was then replaced by the FUS transducer and moved by the stereotactic frame to the targeted brain location using its coordinates in reference to the bregma as determined in reference to the mouse brain atlas.

FIG. 9A is a transverse image of a brain atlas. The desired target location was indicated by a yellow dot in transverse and coronal views of the mouse brain atlas, respectively.

FIG. 9B is a transverse contrast-enhanced MRI image of a mouse brain post-FUS treatment.

FIG. 9C is a transverse image that shows the co-registration of the mouse brain atlas with the MRI image based on anatomic brain structures. The brain atlas is in green, the BBBO area is in purple. The centroid of the BBBO area is indicated by the blue dot.

FIG. 9D is a coronal image of a brain atlas. The desired target location was indicated by a yellow dot in transverse and coronal views of the mouse brain atlas, respectively.

FIG. 9E is a coronal contrast-enhanced MRI image of a mouse brain post-FUS treatment.

FIG. 9F is a coronal image that shows the co-registration of the mouse brain atlas with the MRI image based on anatomic brain structures. The brain atlas is in green, the BBBO area is in purple. The centroid of the BBBO area is indicated by the blue dot.

FIG. 10A is a set of images of simulated ultrasound pressure fields in the transverse and coronal views for 1.5 MHz, 3.0 MHz, and 6.0 MHz FUS transducers, respectively.

FIG. 10B is a set of images from corresponding experimental measurements of the ultrasound pressure fields from the simulations described in FIG. 10A overlaid on illustrations of the mouse brain.

FIG. 10C is a set of graphs of normalized amplitude from simulations (left) and experiments (right) using 1.5 MHz, 3.0 MHz, and 6.0 MHz FUS transducers. (Left) Simulation and (Right) experimental measurement of the beam profiles along axial and lateral directions at each frequency. The shadow in (right) indicates the standard deviation calculated based on measurements performed with three different skulls and each skull with three repeated measurements.

FIG. 10D is a set of graphs of (left) Lateral FWHM diameter, (left center) Axial diameter, (right center) Focal volume of a transducer, and (right) Transcranial transmission ratio with frequency of 1.5, 3.0, 6.0 MHz based on simulated results.

FIG. 10E is a set of graphs of (left) Lateral diameter, (left center) Axial diameter, (right center) Focal volume, and (right) Transcranial transmission ratio with frequencies of 1.5, 3.0, and 6.0 MHz based on experimented results.

FIG. 11A is a graph of the targeting offset along the X-, Y-, and Z-axis for mice treated at 1.5 MHz, 3.0 MHz, and 6.0 MHz.

FIG. 11B is a graph of the absolute Euclidean distance of the targeting offset for different frequencies.

FIG. 11C is a graph of the targeting offset of all groups. Error bars indicate the standard deviation, and ns indicate a nonsignificant difference.

FIG. 12A is a set of transverse (upper) and coronal (bottom) views of CE-MRI images for representative mouse brains treated at different frequencies (1.5, 3.0, or 6.0 MHz) and pressures (0.2, 0.4, or 0.57 MPa).

FIG. 12B is a summary plot of the average Gadolinium delivery volume for all groups. Gadolinium leakage was not detectable in the groups marked by “x”.

FIG. 12C is a graph that shows strong linear correlations between Gadolinium delivery volume and CI. Error bars indicate standard deviation. Shaded areas indicate the 95% confidence band of the linear fitting curves.

FIG. 12D is a graph that shows strong linear correlations between Gadolinium delivery volume and MI. Error bars indicate standard deviation. Shaded areas indicate the 95% confidence band of the linear fitting curves.

FIG. 13A is a set of bright-field (upper) and corresponding fluorescence (bottom) images of coronal sections from representative mouse brains treated at different frequencies (1.5, 3.0, or 6.0 MHz) and pressures (0.2, 0.4, or 0.57 MPa) after Evans Blue administration.

FIG. 13B is a summary plot of the average Evens blue volume and signal intensity and for all groups. Evans blue was not detectable in the groups marked by “x”.

FIG. 13C is a graph of the correlation between Evans blue volume and CI. The equation shows the linear regression of the fitted curve, and EB represents Evans blue. Error bars indicate standard deviation. Shaded areas indicate a 95% confidence band of the fitting curves.

FIG. 13D is a graph of the correlation between Evans blue volume and MI. The equation shows the linear regression of the fitted curve, and EB represents Evens blue. Error bars indicate standard deviation. Shaded areas indicate a 95% confidence band of the fitting curves.

FIG. 13E is a graph of the correlation between Evans blue signal intensity and CI. The equation shows the linear regression of the fitted curve, and EB represents Evens blue. Error bars indicate standard deviation. Shaded areas indicate a 95% confidence band of the fitting curves.

FIG. 13F is a graph of the correlation between Evans blue signal intensity and MI. The equation shows the linear regression of the fitted curve, and EB represents Evens blue. Error bars indicate standard deviation. Shaded areas indicate a 95% confidence band of the fitting curves.

FIG. 14A is an H&E-stained brain section after FUS exposure at 0.57 MPa with 1.5 MHz. Hemorrhage was observed, as shown by the high-magnification images.

FIG. 14B is an H&E-stained brain section after FUS exposure at 0.57 MPa with 3.0 MHz. Hemorrhage was not observed, as shown by the high-magnification images.

FIG. 14C is an H&E-stained brain section after FUS exposure at 0.57 MPa with 6.0 MHz. Hemorrhage was not observed, as shown by the high-magnification images.

### DETAILED DESCRIPTION OF THE INVENTION

In various aspects, an affordable and easy-to-use FUS device for spatially accurate and precise FUS-BBBO is disclosed.

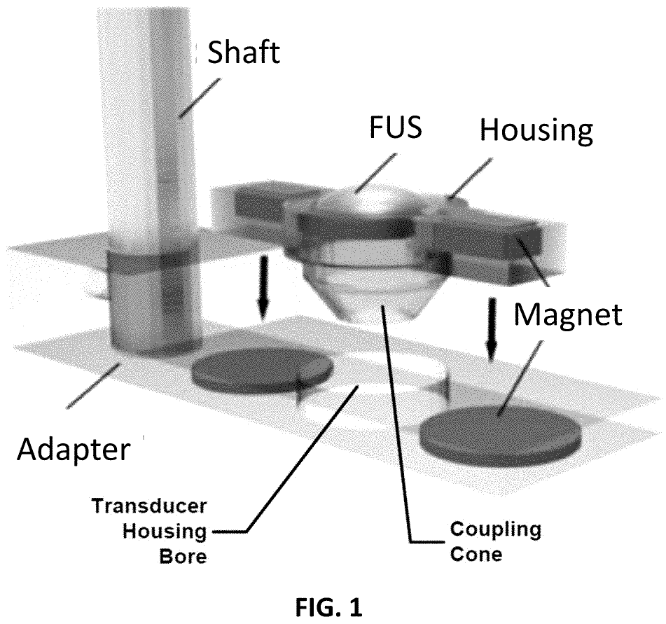

FIG. 1 shows the elements of a FUS device in one aspect. The FUS device includes an adapter, a housing, a focused ultrasound transducer (FUS), and a transducer housing. The adapter is configured to be attached to a shaft or other actuated mount of a stereotactic device. The transducer housing is configured to receive the ultrasound transducer and to reversibly couple to the adapter. In various aspects, the transducer housing may be coupled to the adapter using any suitable fasteners or other coupling means without limitation including, but not limited to, screws, clamps, pins, adhesives, magnets, and any other suitable coupling devices or methods. In one aspect, the transducer housing is coupled to the adapter using paired magnets positioned in the transducer housing and adapter such that magnetic attractive forces are generated when the transducer housing is positioned sufficiently close to the adapter, causing the transducer housing to couple to the adapter via magnetic forces.

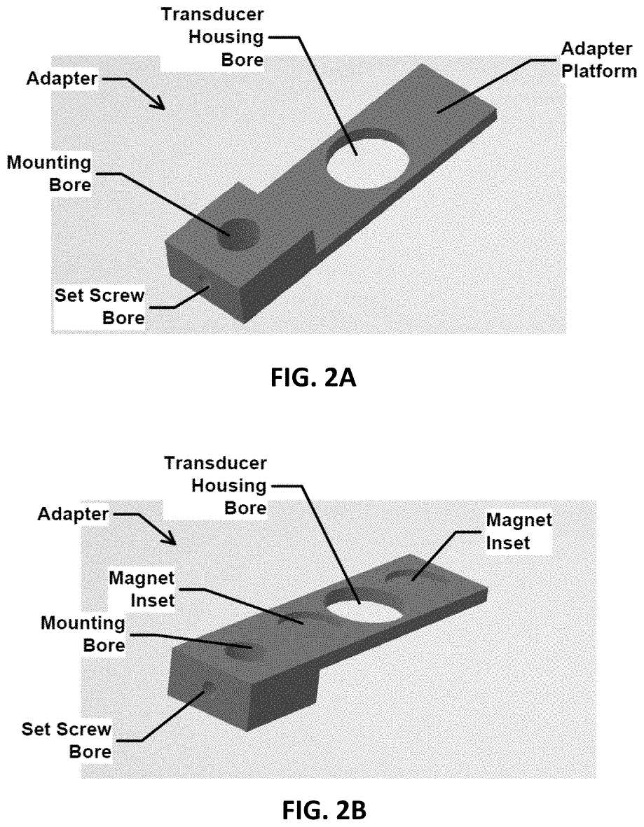

FIGS. 2A and 2B illustrate an adapter in one aspect. The adapter includes an adapter platform that defines a mounting bore at one end configured to receive a mounting shaft of a stereotaxic system, as illustrated in FIG. 1. The mounting shaft of the stereotaxic system is configured to be positioned with high precision using the actuators of the stereotactic system. In various aspects, the adapter provides for high-precision positioning of the transducer to facilitate focused ultrasound-mediated blood-brain barrier (BBB) opening (FUS-BBBO). Referring again to FIGS. 2A and 2B, the adapter platform further defines a set screw bore passing through an end of the adapter platform into the mounting bore. The set screw bore is configured to receive a set screw used to couple the adapter to the mounting shaft, as shown in FIG. 5.

Referring again to FIGS. 2A and 2B, the adapter platform further defines a transducer housing bore configured to receive the transducer housing as described in additional detail below. In some aspects, the adapter platform further includes at least one magnet inset configured to receive and house a magnet used to couple the transducer housing to the adapter platform as described in additional detail below.

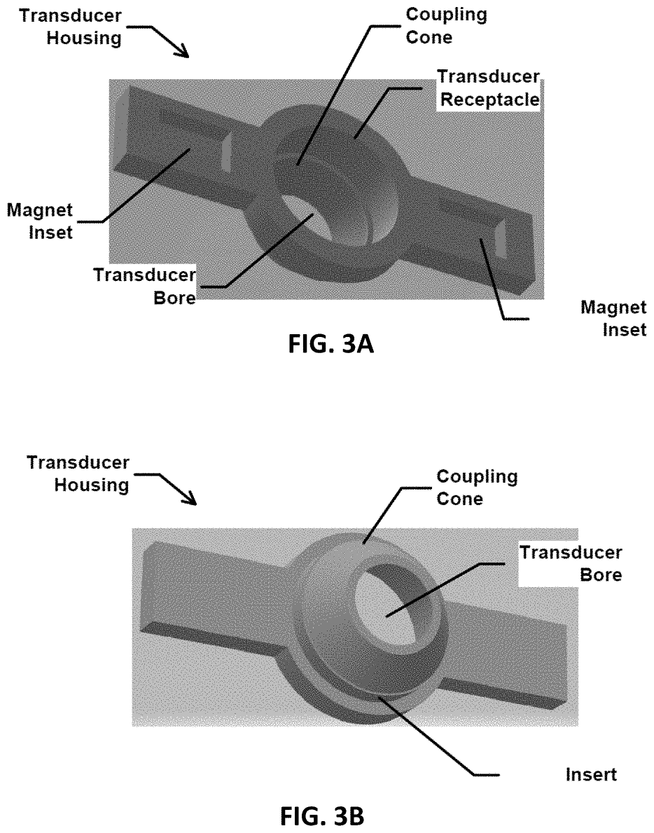

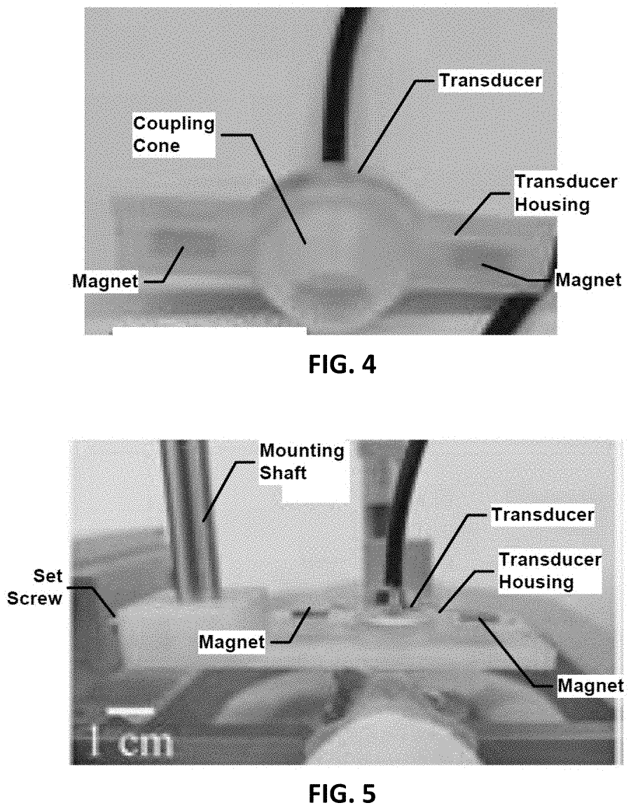

In various aspects, the FUS device further includes a transducer housing configured to receive and house a focused ultrasound (FUS) transducer and to reversibly couple to the adapter. As illustrated in FIGS. 3A and 3B, the transducer housing includes a transducer bore connecting a transducer receptacle and a coupling cone positioned on opposite faces of the transducer housing. The transducer receptacle is dimensioned to receive and house the FUS transducer, as illustrated in FIG. 4. In various aspects, the transducer receptacle provides for the removal and replacement of FUS transducers as needed to replace a malfunctioning FUS transducer, to substitute a first FUS transducer for a second FUS transducer with different operational characteristics, or as needed for any other system requirement without limitation.

Referring again to FIGS. 3A and 3B, the coupling cone projects downward from the transducer bore. In various aspects, the coupling cone is configured to contain an impedance-matched ultrasound coupling material including, but not limited to, ultrasound gel to enhance ultrasound coupling of the FUS transducer to the targeted tissues when in use for FUS-BBBO.

In various aspects, at least one magnet inset configured to receive and house at least one magnet is defined within the transducer housing, as illustrated in FIG. 3A. In various aspects, the transducer housing is reversibly coupled to the adapter platform by inserting the coupling cone of the transducer housing through the transducer housing bore of the adapter and aligning the magnets housed in the transducer housing and adapter platform, respectively, as illustrated in FIG. 1. FIG. 5 shows the transducer housing and FUS transducer coupled to the adapter platform.

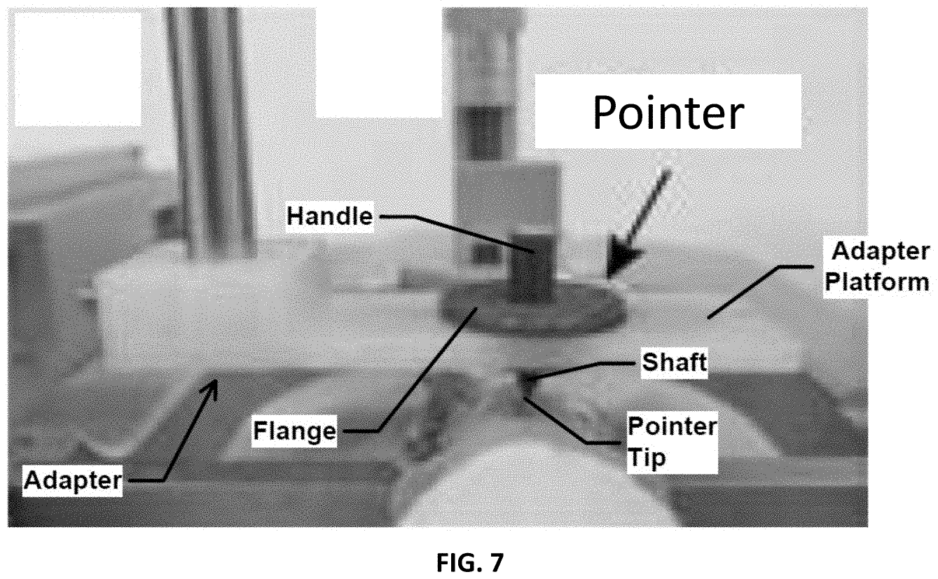

In various additional aspects, the FUS device further includes a pointer insert for positioning the adapter platform in an initial position prior to mounting the FUS transducer and transducer housing. The pointer is configured to fit within the transducer housing bore of the adapter (FIG. 2A), as illustrated in FIG. 7. In some aspects, when the pointer is positioned within the transducer housing, the pointer tip is aligned with the path of the focused ultrasound pulses produced by the FUS transducer. In various aspects, the pointer tip is moved into alignment with a predetermined target or landmark by adjusting the position of the platform using the stereotaxic system.

In some aspects, the landmark to which the pointer tip is aligned includes, but is not limited to, an external feature of the FUS-BBBO subject. In other aspects, the external feature used as a landmark marks a desired location for a FUS-BBO treatment on a subject. In other aspects, the external feature provides an origin or reference point. In these other aspects, the platform may be repositioned relative to the origin or reference point according to a surgical map or atlas describing the position of the desired treatment location relative to the origin or reference point. By way of non-limiting example, the bregma of a mouse, which is visible through the mouse's shaved scalp, may be selected as an origin or reference point to which the pointer tip is aligned, after which specific brain structure of the mouse may be located by moving the platform and attached transducer relative to the bregma as specified by a mouse brain atlas.

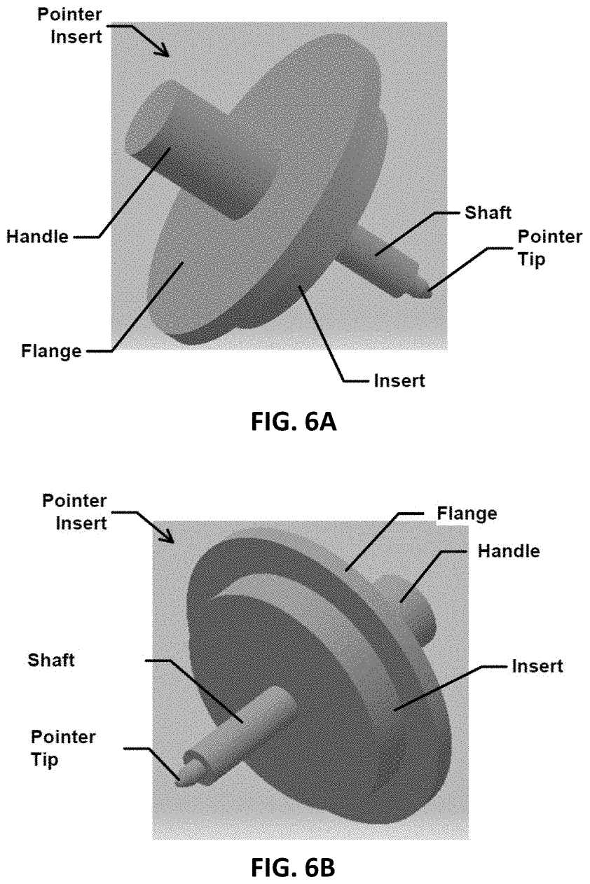

Referring to FIGS. 6A and 6B, the pointer insert includes a flange with an upward-projecting handle and a downward-projecting insert. The insert is sized and dimensioned to fit snugly within the transducer housing bore of the adapter, and the flange is sized and dimensioned slightly larger than the transducer housing bore to rest against the adapter platform and retain the pointer insert in a fixed position relative to the adapter (see FIG. 7). Referring again to FIGS. 6A and 6B, the pointer insert further includes a shaft projecting downward from the insert and ending in a pointer tip. In various aspects, the pointer tip provides a visual aid to align the direction of the FUS transducer to an intended target for FUS-BBBO as described above.

By way of non-limiting example, a stereotactic-guided FUS system (FIG. 1) includes a commercially available stereotaxic apparatus and a FUS device attached to an actuated shaft of the stereotaxic apparatus. The FUS device includes at least one in-house manufactured miniature FUS transducer, a 3D-printed transducer housing and adapter, and a transducer driving system that includes a commercially available function generator (Model 33500B, Keysight Technologies Inc., Englewood, Colo., USA) and a power amplifier (1020L, Electronics & Innovation, Rochester, N.Y., USA). The stereotaxic apparatus (Model 940, David Kopf Instruments, Tujunga, Calif.) has a 10-micron movement resolution for all axes and includes an easy-to-read compact digital display console to facilitate positioning the FUS device over a targeted treatment location.

The FUS transducers of this example were miniature FUS transducers that had an aperture of 13 mm and a focal length of 10 mm. The FUS transducers included lead zirconate titanate (PZT) ceramic piezo material (DL-47, Del Piezo Specialties LLC, West Palm Beach, Fla., USA). These miniaturized transducers provided sufficient output pressure because PZT is a material commonly used material for high-power ultrasound transmission. As described in the Examples herein, FUS transducers producing three different frequencies (1.5 MHz, 3 MHz, and 6 MHz) were included to investigate the relationship between frequency and BBBO outcome. The manufacturing of these transducers was straightforward as it only required gluing two wires to the positive and negative electrodes of the piezoelectric element.

In this non-limiting example, the transducer element was encapsulated in the 3D-printed transducer housing using epoxy (Devcon Epoxy Adhesive, Devcon Corp., Danvers, Mass.). The back of the transducer directly contacted the air to form air backing. The transducers were then connected through these wires to a power amplifier coupled with a function generator. No electrical impedance matching was needed because the real part of the transducer impedance at the resonance frequency as measured by an E5061A ENA Network Analyzer with the 85070E Dielectric Probe Kit (Agilent Technologies, Santa Clara, Calif., USA) was in the range of 31 to 59 ohms, which was close to 50 ohms needed for a perfect impedance match.

The transducer housing, shown in FIG. 4, was manufactured using a 3D printer (Ultimaking Ltd., Netherlands). The housing included a coupling cone, which was filled with ultrasound gel when in use to enhance acoustic coupling. Two pairs of magnets (FIG. 5) were used to enable simple attachment and detachment of each transducer housing from the adaptor. The adaptor connects the FUS transducer to the actuated mounting shaft of a stereotaxic system commonly used to position needles for stereotactic injections.

In order to achieve precise targeting of the FUS transducer at a specific brain location, a pointer was manufactured by 3D printing (FIGS. 6A and 6B). The tip of the pointer indicated the geometrical focus of the FUS transducer. The procedure for aligning the FUS transducer to target a specific brain location was similar to the established stereotactic procedure. A dot was drawn on the mouse's scalp to indicate the location of the bregma, which was visible through the scalp. The pointer was then placed in the platform, and its position was adjusted by the stereotactic frame to align with the dot. Once the pointer was properly positioned, the pointer insert was replaced in the adapter by the FUS transducer in the transducer housing (FIG. 5). The platform was then moved to position the FUS transducer over a predetermined target location using its coordinates in reference to the bregma as determined in reference to the mouse brain atlas.

As illustrated in the Examples herein, the disclosed FUS device achieved FUS-BBBO with sub-millimeter accuracy as measured by the offset between the desired target location and the BBBO centroid. Overall, the present disclosure describes an affordable and easy-to-use FUS device for spatially accurate, precise, and tunable drug delivery to the mouse brain.

The FUS device can have the following features: (1) The FUS transducer elements are widely available at a low cost (˜$80 per element). Transducer manufacturing only required connecting wires to the electrodes on the elements. All other components can be 3D printed. (2) The integration of the FUS transducers with a stereotactic frame for targeting desired brain location using established stereotactic procedures decreased the barrier to the adoption of the FUS technique. The device achieved sub-millimeter targeting accuracy. (3) The use of higher frequency FUS transducers (3 MHz and 6 MHz) decreased the BBBO volume and improved the spatial precision of FUS-BBBO in targeting individual structures in the mouse brain. (4) The drug delivery outcome was tunable by adjusting the CI or MI. In some embodiments, the device may be manufactured by research groups without an ultrasound background and used in various applications with minimal training needed.

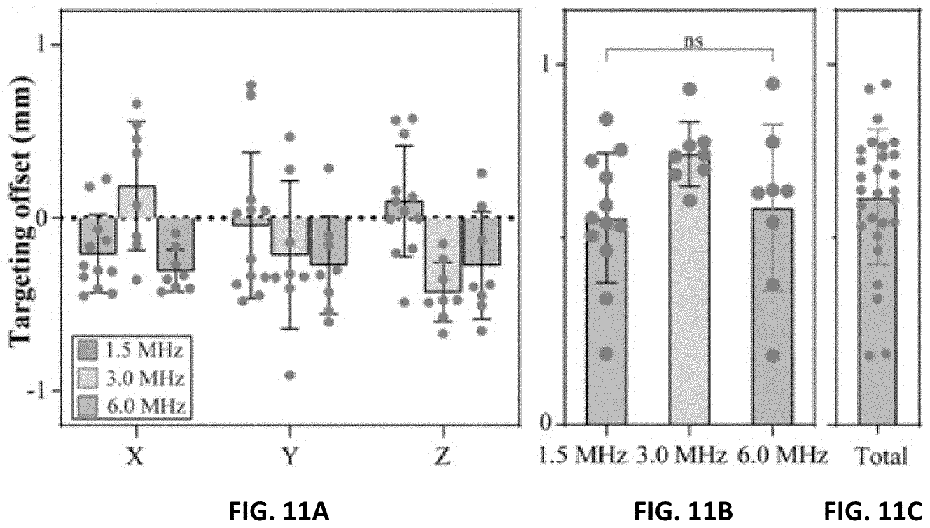

In an exemplary embodiment, the targeting accuracy of the stereotactic-guided FUS system was high, reaching 0.63±0.19 mm (FIG. 11C). The primary step that determined the targeting accuracy was the alignment of the pointer with the bregma. Although extensive preclinical FUS-BBBO studies have been reported, few studies reported targeting accuracy. The only report we found was by Bing et al., who used a stereotactic-guided FUS system and reported a targeting accuracy of ±0.3 mm in the rat brain based on the region of Evans blue staining on ex vivo gross sections. The accuracy of the system of the present disclosure was comparable to theirs and the current quantification method based on in vivo CE-MRI provided was a more reliable measurement of the targeting accuracy than the ex vivo measurements based on Evans blue leakage.

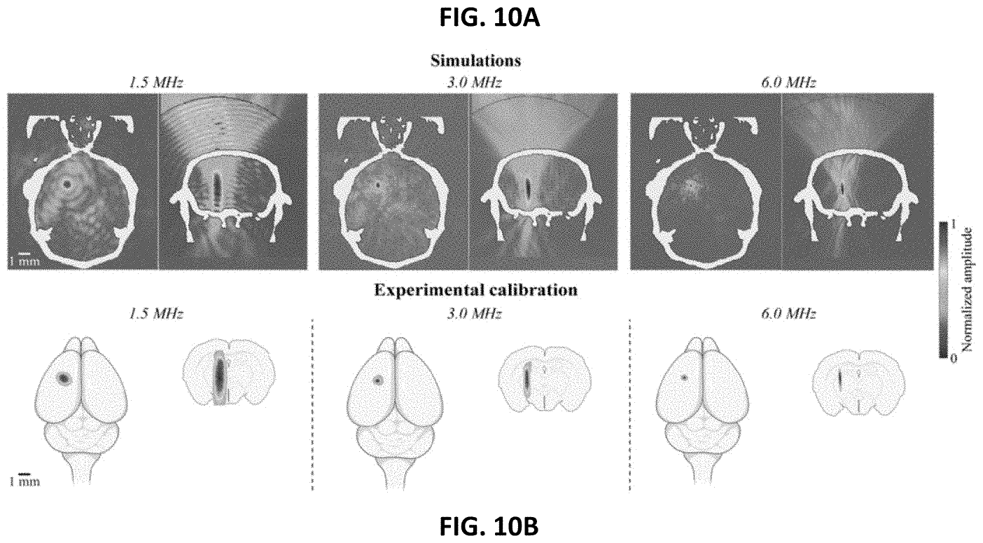

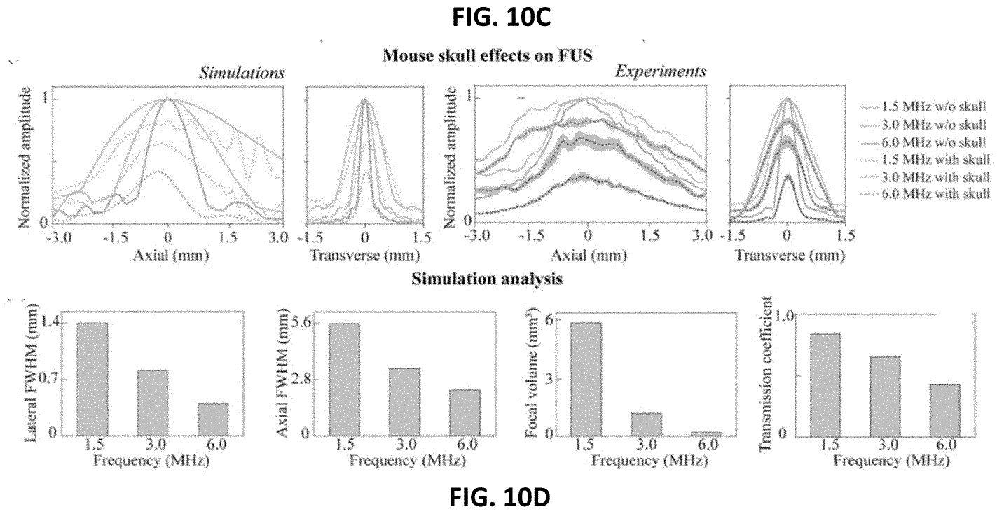

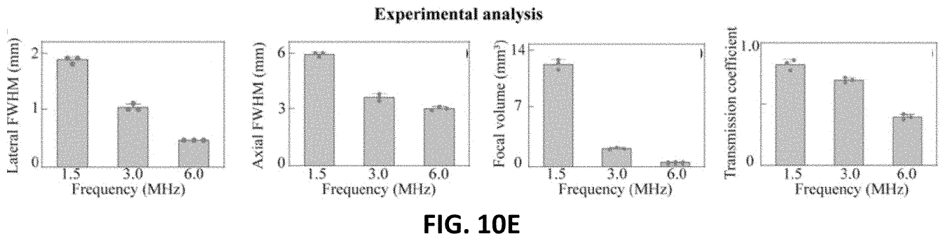

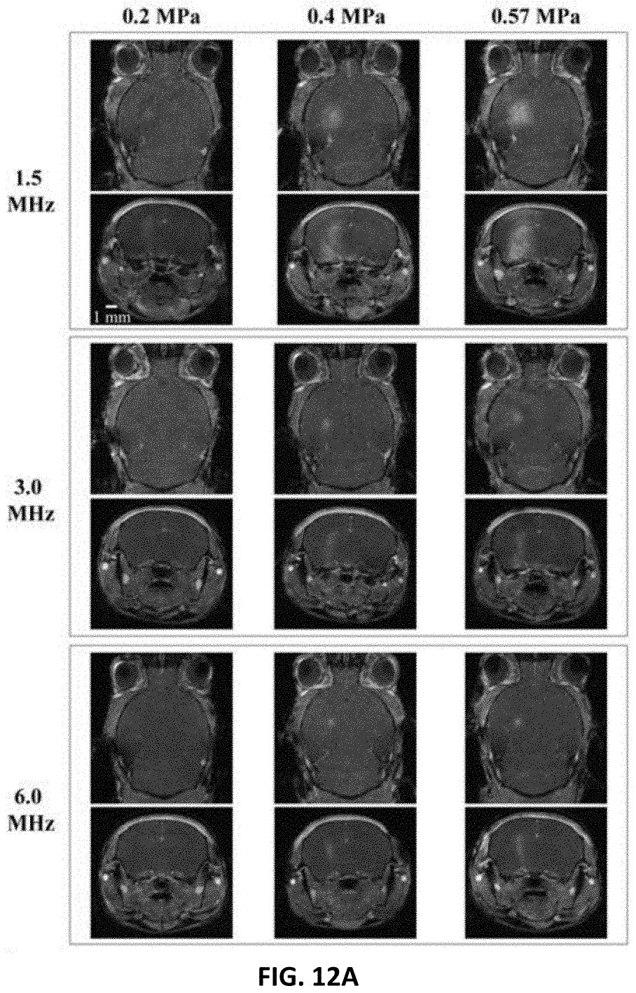

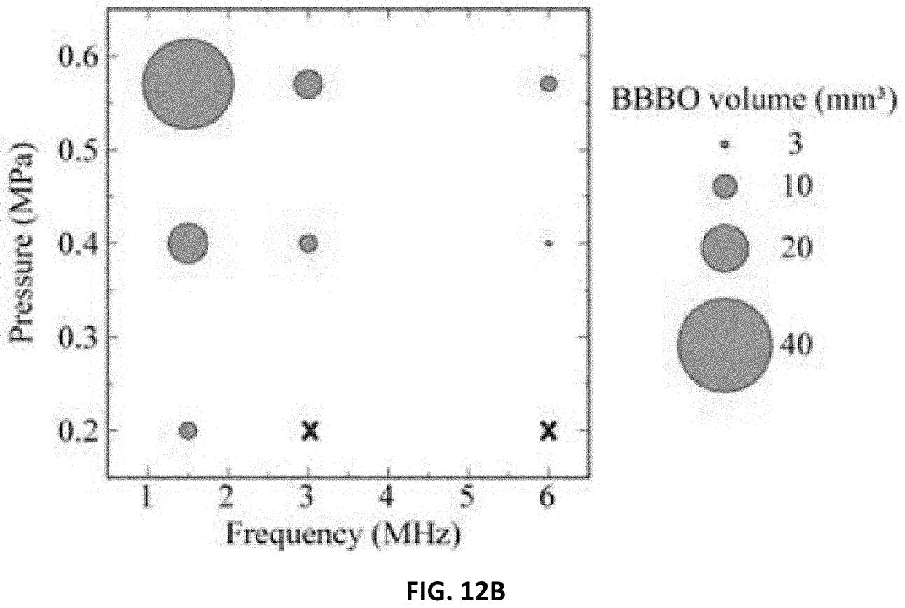

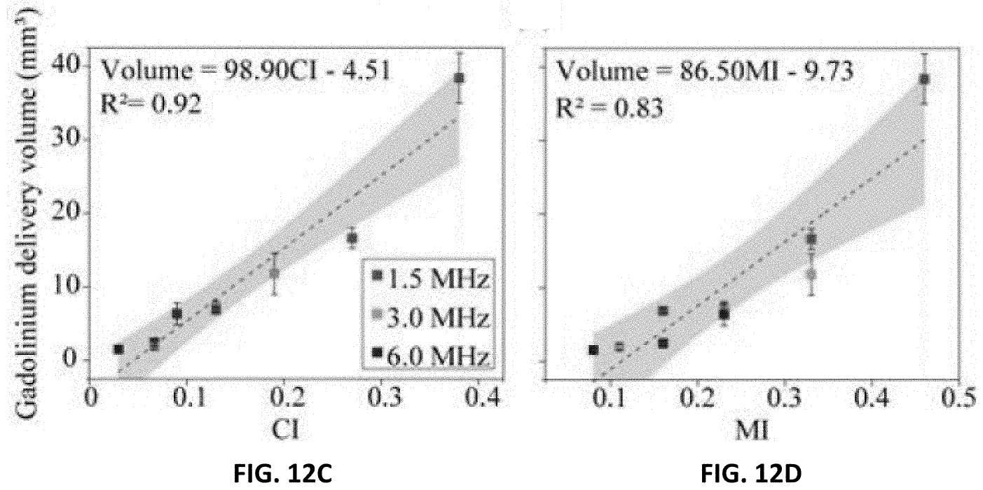

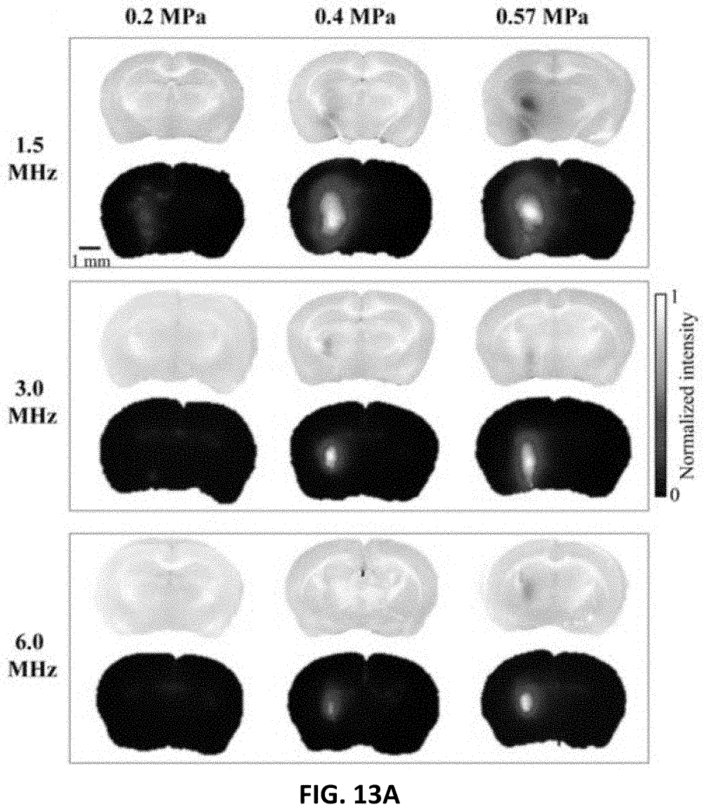

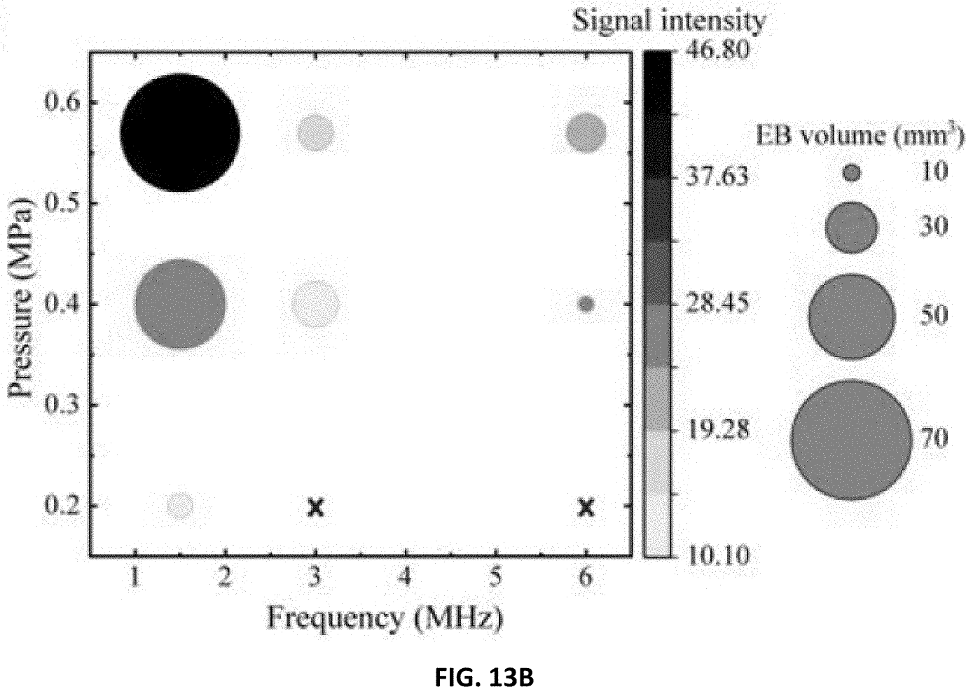

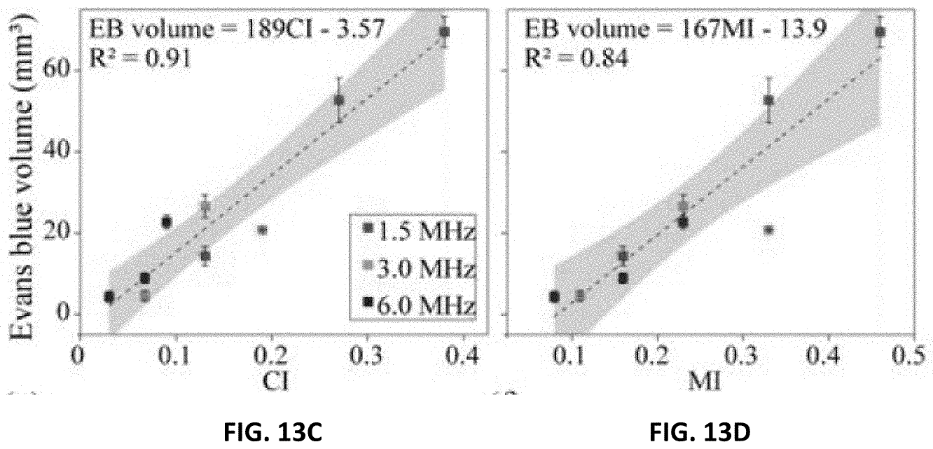

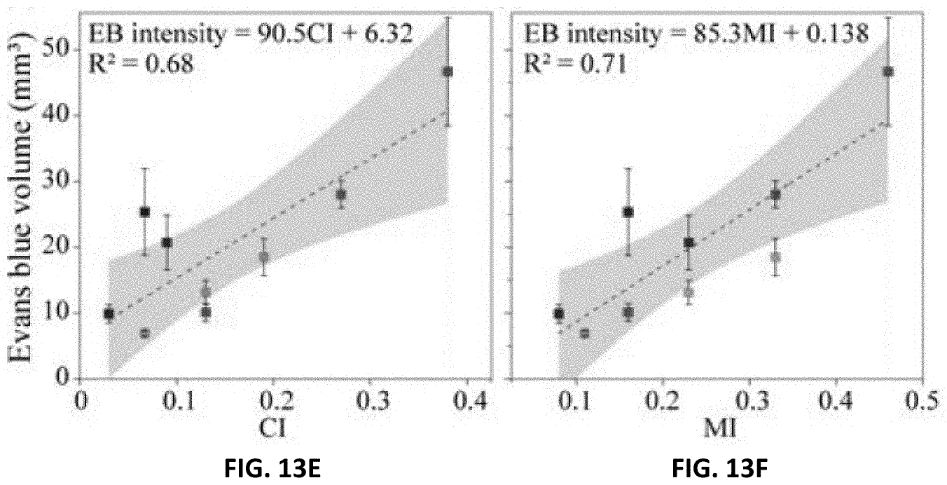

Multiple strategies have been proposed to improve the spatial precision of FUS in achieving BBBO in individual brain structures. One strategy is to replace a single-element FUS transducer with a large-aperture phased array, but the high cost and complexity of the phased array limit its broad adoption. Other strategies include the use of two transducers with frequency-modulated crossed beams and the use of chirp and random frequency-modulated ultrasound waveforms. The focal region size of a single-element FUS transducer can be approximately estimated using λF # in the lateral direction and 7λ(F #)² in the axial direction, where λ is the wavelength and F #=focal length/aperture. It is well-known that increasing the transducer frequency can decrease the focal region size. It is demonstrated herein that under the same pressure level, a higher frequency FUS transducer achieved a small drug delivery volume (FIGS. 12 and 13), which improved the spatial precision of FUS-BBBO compared with that achieved with lower frequency transducers. There is a potential concern that the skull can distort the beam at higher frequencies, however, it was found that a focused beam pattern was formed even in the presence of the skull at all frequencies tested (FIGS. 10A and B). The mouse skull only lead to <0.3 mm shift in the axial direction and <0.1 mm in the lateral direction at all three frequencies based on simulations (FIG. 10C). Although the skull contributed to 17.0-61% attenuation of the acoustic pressure at 1.5-6.0 MHz, the skull attenuation was easily compensated by increasing the amplitude of the driving signal. The finding that the targeting accuracy of the FUS device was independent of the FUS frequency (FIG. 11) further indicated that the effect of the skull did not lead to a significant shift of the focal point. It is worth pointing out that, if needed, new FUS transducers can be designed with lower F # to further decrease the focal region size

The FUS-BBBO drug delivery outcome can be tuned by the CI and MI. Strong linear correlations were found between Gadolinium delivery volume and CI or MI (FIG. 12). Similar strong linear correlations were also found between Evans blue delivery volume and CI or MI and relatively lower correlations were found between the Evans blue signal intensity and CI or MI (FIG. 13). Using two FUS transducers at 0.4 MHz and 1 MHz, one previous study also found strong linear correlations between MI/CI and CE-MRI signal intensity changes. The current findings suggest that these strong correlations can be expanded to FUS-BBBO at higher frequencies, further confirming that CI and MI can be used to predict the FUS-BBBO drug delivery outcome. These findings also indicate that users can perform Evans blue delivery to calibrate their FUS devices. FUS devices are normally calibrated with a hydrophone, as reported in our study. However, hydrophone calibration requires dedicated devices to obtain measurement data and further requires knowledge of acoustics for processing the data. In contrast, Evans blue is cheap and widely available. Users without knowledge of acoustics can perform calibration of the FUS device using Evans blue. They can establish the correlations between different FUS parameters and Evans blue delivery outcome and benchmark with our findings. When using this device for different applications, users can use the established correlations to select the FUS transducer and acoustic pressure based on the intended drug delivery outcome. It needs to point out that the FUS-BBBO drug delivery outcome is dependent on the properties of the agents (e.g., type of agents, molecular weight, and surface charge). Evans blue can be used as a model agent to calibrate the FUS device, but the delivery outcome of different agents is expected to be different.

The methods and algorithms of the invention may be enclosed in a controller or processor. Furthermore, methods and algorithms of the present invention, can be embodied as a computer-implemented method or methods for performing such computer-implemented method or methods, and can also be embodied in the form of a tangible or non-transitory computer-readable storage medium containing a computer program or other machine-readable instructions (herein “computer program”), wherein when the computer program is loaded into a computer or other processor (herein “computer”) and/or is executed by the computer, the computer becomes an apparatus for practicing the method or methods. Storage media for containing such computer programs include, for example, floppy disks and diskettes, compact disk (CD)-ROMs (whether or not writeable), DVD digital disks, RAM and ROM memories, computer hard drives and backup drives, external hard drives, “thumb” drives, and any other storage medium readable by a computer. The method or methods can also be embodied in the form of a computer program, for example, whether stored in a storage medium or transmitted over a transmission medium such as electrical conductors, fiber optics or other light conductors, or by electromagnetic radiation, wherein when the computer program is loaded into a computer and/or is executed by the computer, the computer becomes an apparatus for practicing the method or methods. The method or methods may be implemented on a general-purpose microprocessor or on a digital processor specifically configured to practice the process or processes. When a general-purpose microprocessor is employed, the computer program code configures the circuitry of the microprocessor to create specific logic circuit arrangements. Storage medium readable by a computer includes medium being readable by a computer per se or by another machine that reads the computer instructions for providing those instructions to a computer for controlling its operation. Such machines may include, for example, machines for reading the storage media mentioned above.

Definitions and methods described herein are provided to better define the present disclosure and to guide those of ordinary skill in the art in the practice of the present disclosure. Unless otherwise noted, terms are to be understood according to conventional usage by those of ordinary skill in the relevant art.

In some embodiments, numbers expressing quantities of ingredients, properties such as molecular weight, reaction conditions, and so forth, used to describe and claim certain embodiments of the present disclosure are to be understood as being modified in some instances by the term “about.” In some embodiments, the term “about” is used to indicate that a value includes the standard deviation of the mean for the device or method being employed to determine the value. In some embodiments, the numerical parameters set forth in the written description and attached claims are approximations that can vary depending upon the desired properties sought to be obtained by a particular embodiment. In some embodiments, the numerical parameters should be construed in light of the number of reported significant digits and by applying ordinary rounding techniques. Notwithstanding that the numerical ranges and parameters setting forth the broad scope of some embodiments of the present disclosure are approximations, the numerical values set forth in the specific examples are reported as precisely as practicable. The numerical values presented in some embodiments of the present disclosure may contain certain errors necessarily resulting from the standard deviation found in their respective testing measurements. The recitation of ranges of values herein is merely intended to serve as a shorthand method of referring individually to each separate value falling within the range. Unless otherwise indicated herein, each individual value is incorporated into the specification as if it were individually recited herein. The recitation of discrete values is understood to include ranges between each value.

In some embodiments, the terms “a” and “an” and “the” and similar references used in the context of describing a particular embodiment (especially in the context of certain of the following claims) can be construed to cover both the singular and the plural, unless specifically noted otherwise. In some embodiments, the term “or” as used herein, including the claims, is used to mean “and/or” unless explicitly indicated to refer to alternatives only or the alternatives are mutually exclusive.

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition or device that “comprises,” “has” or “includes” one or more features is not limited to possessing only those one or more features and can cover other unlisted features.

All methods described herein can be performed in any suitable order unless otherwise indicated herein or otherwise clearly contradicted by context. The use of any and all examples, or exemplary language (e.g., “such as”) provided with respect to certain embodiments herein is intended merely to better illuminate the present disclosure and does not pose a limitation on the scope of the present disclosure otherwise claimed. No language in the specification should be construed as indicating any non-claimed element essential to the practice of the present disclosure.

Groupings of alternative elements or embodiments of the present disclosure disclosed herein are not to be construed as limitations. Each group member can be referred to and claimed individually or in any combination with other members of the group or other elements found herein. One or more members of a group can be included in, or deleted from, a group for reasons of convenience or patentability. When any such inclusion or deletion occurs, the specification is herein deemed to contain the group as modified thus fulfilling the written description of all Markush groups used in the appended claims.

All publications, patents, patent applications, and other references cited in this application are incorporated herein by reference in their entirety for all purposes to the same extent as if each individual publication, patent, patent application, or other reference was specifically and individually indicated to be incorporated by reference in its entirety for all purposes. Citation of a reference herein shall not be construed as an admission that such is prior art to the present disclosure.

Having described the present disclosure in detail, it will be apparent that modifications, variations, and equivalent embodiments are possible without departing from the scope of the present disclosure defined in the appended claims. Furthermore, it should be appreciated that all examples in the present disclosure are provided as non-limiting examples.

### EXAMPLES

The following non-limiting examples are provided to further illustrate the present disclosure. It should be appreciated by those of skill in the art that the techniques disclosed in the examples that follow represent approaches the inventors have found function well in the practice of the present disclosure, and thus can be considered to constitute examples of modes for its practice. However, those of skill in the art should, in light of the present disclosure, appreciate that many changes can be made in the specific embodiments that are disclosed and still obtain a like or similar result without departing from the spirit and scope of the present disclosure.

### Example 1—Design and Fabrication of the FUS Device

The objective of this study was to design and fabricate an affordable and easy-to-use FUS device for spatially accurate and precise FUS-BBBO. Mini-FUS transducers ($80 each in material cost) with different frequencies (1.5, 3.0, and 6.0 MHz) were manufactured in-house and integrated with a commercially available stereotactic frame using 3D-printed parts. It was found that this device achieved FUS-BBBO with sub-millimeter accuracy as measured by the offset between the desired target location and the BBBO centroid. It was also shown that FUS-BBBO volume could be decreased to perform spatially precise BBBO. The drug delivery volume and signal intensity were tunable by the CI and MI.

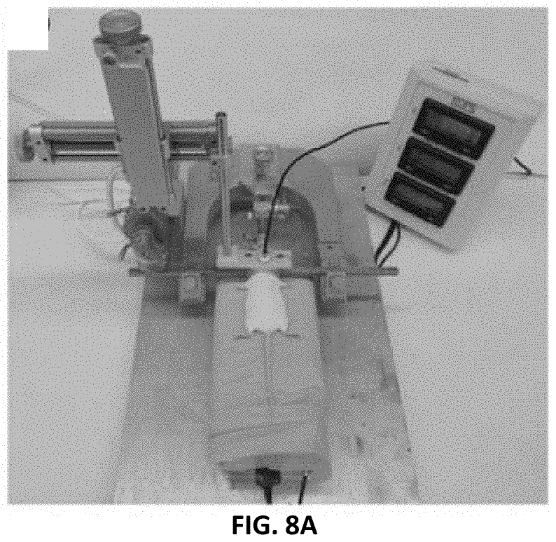

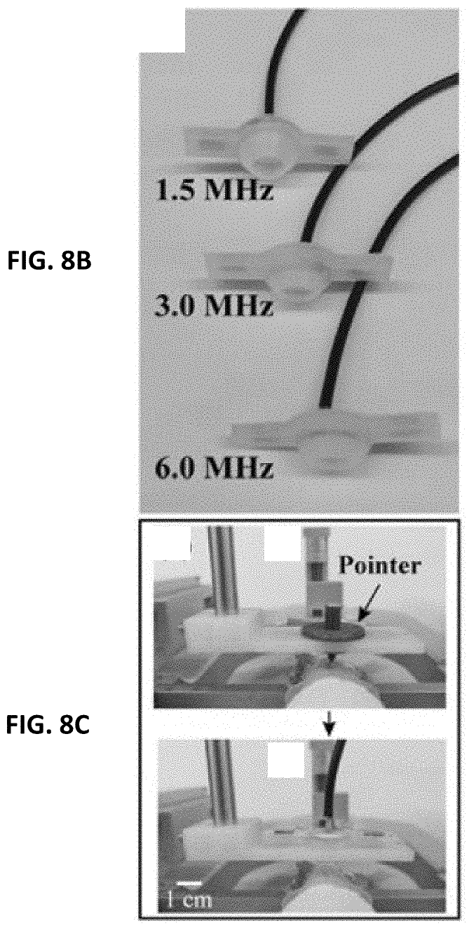

The stereotactic-guided FUS device (FIG. 8A) consisted of a commercially available stereotaxic apparatus; in-house manufactured miniature FUS transducers; 3D-printed transducer housing and adapter; and transducer driving system, including a commercially available function generator (Model 33500B, Keysight Technologies Inc., Englewood, Colo., USA) and a power amplifier (1020L, Electronics & Innovation, Rochester, N.Y., USA). The stereotaxic apparatus (Model 940, David Kopf Instruments, Tujunga, Calif.) have been widely used in neuroscience for small animal research. It has a 10-micron movement resolution for all axes and includes an easy-to-read compact digital display console.

The key hardware component was the FUS transducer. The miniature FUS transducers had an aperture of 13 mm and a focal length of 10 mm. They were made by lead zirconate titanate (PZT) ceramic piezo material (DL-47), which was purchased from Del Piezo Specialties LLC. (West Palm Beach, Fla., USA) at a cost of ˜$80 per element. These miniaturized transducers could provide sufficient output pressure because PZT is the most commonly used material for high-power ultrasound transmission. Elements with three different frequencies were chosen (FIG. 8B), 1.5 MHz, 3 MHz, and 6 MHz, to investigate the relationship between frequency and BBBO outcome. The manufacturing of these transducers was straightforward as it only required gluing two wires to the positive and negative electrodes of the piezoelectric element.

The element was then encapsulated in a 3D-printed housing using epoxy (Devcon Epoxy Adhesive, Devcon Corp., Danvers, Mass.). The back of the transducer directly contacted the air to form air backing. The transducers were then connected through these wires to a power amplifier coupled with a function generator. No electrical impedance matching was needed because the real part of the transducer impedance at the resonance frequency as measured by an E5061A ENA Network Analyzer with the 85070E Dielectric Probe Kit (Agilent Technologies, Santa Clara, Calif., USA) was in the range of 31 to 59 ohms, which was close to 50 ohms needed for a perfect impedance match.

The design of the transducer housing is shown in FIG. 1, which was manufactured using a 3D printer (Ultimaking Ltd., Netherlands). The housing provided a coupling cone, which was filled with ultrasound gel when in use for acoustic coupling. Two pairs of magnets were used to enable simple attach and detach of the transducer from the adaptor. The adaptor connects the FUS transducer to the bar commonly used to hold needles for stereotactic injections.

In order to achieve precise targeting of the FUS transducer at a specific brain location, a pointer was manufactured by 3D printing (FIG. 8C, top). The tip of the pointer indicated the geometrical focus of the FUS transducer. The procedure for aligning the FUS transducer to target a specific brain location was similar to the established stereotactic procedure. First, a dot was drawn on the mouse's scalp to indicate the location of the bregma, which was visible through the scalp. The pointer was then placed in the holder, and its position was adjusted by the stereotactic frame to align with the dot (FIG. 8C, top). Second, the pointer was switched to the FUS transducer and was moved to the target location using its coordinates in reference to the bregma as determined in reference to the mouse brain atlas (FIG. 8C, bottom).

### Example 2—Simulation and Calibration of the FUS Transducers

To assess the performance of the disclosed FUS device, the following experiments were conducted. The acoustic pressure fields generated by the FUS transducers in a mouse head were simulated using a k-space pseudospectral method-based solver (Matlab toolbox, k-Wave). Experimental calibration of each FUS transducer was performed in degassed water without and with three degassed ex vivo mouse skulls.

### Methods

The acoustic pressure fields generated by the FUS transducers in a mouse head were simulated using a k-space pseudospectral method-based solver (Matlab toolbox, k-Wave). A mouse head was placed in a μCT scanner (Rigaku, Tokyo, Japan). The acquired CT images consisted of 512×512×679 voxels with a spatial resolution of 0.08 mm. Linear interpolation was performed to adjust the voxel spacing of the images to 0.12 mm for 1.5 MHz, 0.06 mm for 3 MHz, and 0.03 mm for 6 MHz, to ensure the voxel spacing was less than ⅛ of the corresponding FUS transducer's wavelength. The density and sound speed of the skull and brain tissue were converted from the Hounsfield units of the CT images using the function ‘hounsfield2density’ in the k-Wave toolbox. This function uses a piecewise linear fit to the data reported by Schneider and Mast. The sound speed of the coupling gel was set to be the same as that of the water (1484 m/s). A Courant-Friedrichs-Lewy (CFL) stability factor of 0.17 was used in all the simulations. Experimental calibration of each FUS transducer was performed in degassed water without and with three degassed ex vivo mouse skulls. The skulls were positioned in front of the transducer. The acoustic pressure fields were measured using a hydrophone (HGL-200, ONDA Corporation, Sunnyvale, Calif.), which was moved in 3D using a computer-controlled 3D stage (PK245-01AA, Velmex Inc., NY, USA). The transmission efficiency of each FUS transducer was measured by the ratio between the maximum peak negative pressures measured with and without the skull.

### Results

FIG. 10A shows the transverse and coronal views of the simulated acoustic pressure fields at frequencies of 1.5, 3.0, and 6.0 MHz, respectively. The focal region sizes defined by the full width at half maximum (FWHM) in the axial and lateral directions are shown in FIG. 10C. FIG. 10B displays the experimental measurement results with the mouse skull, and the measured focal region sizes are presented in FIG. 10D. FIG. 10C shows the beam profiles along the axial and lateral directions obtained from simulations (FIG. 10C (left)) and experiments (FIG. 10C (left center)). The simulation results showed that the FUS focus was shifted by 0.3 mm along the axial direction toward the transducer at 6.0 MHz and 0.2 mm at 3.0 MHz. The shift along the lateral direction was within 0.1 mm for all frequencies. Based on experimental measurement with only the top piece of the mouse skull, mouse skulls only lead to less than 0.1 mm shift of the FUS focus in the axial and lateral directions at all frequencies. No standing wave formed at 1.5 MHz due to the presence of the hydrophone. As the FUS frequency increased from 1.5 MHz to 6.0 MHz, the FWHM in the axial direction decreased from 5.6 mm, 3.4 mm, to 2.2 mm based on the simulation results (FIG. 10D (left) and from 5.9±0.1 mm, 3.6±0.2 mm, to 3.0±0.1 mm according to the experimental measurements (FIG. 10E (left). The FWHM in the lateral direction decreased from 1.4 mm, 0.8 mm, to 0.4 mm in simulation (FIG. 10D (left center)) and from 1.8±0.1 mm, 1.0±0.1 mm, to 0.5±0.0 mm in calibration (FIG. 10E (left center)). The calculated focal volume based on the ellipsoid volume equation (V=4π/3 ab², wherein a is the half axial FWHM, b is the half transverse FWHM) decreased from 5.7 mm³, 1.2 mm³, to 0.2 mm³ in simulation (FIG. 10D (right center)) and from 11.2±0.7 mm³, 2.0±0.1 mm³, to 0.3±0.0 mm³ in calibration (FIG. 10E (right center)). The FUS transmission coefficient was 84.3% for 1.5 MHz, 65.4% for 3.0 MHz, 42.7% for 6.0 MHz based on simulation (FIG. 10D (right)), and 81.0%±4.2% for 1.5 MHz, 65.2%±2.1% for 3.0 MHz, and 38.6%±2.2% for 6.0 MHz based on calibration (FIG. 10E (right)).

### Example 3—In Vivo Evaluation of Targeting Accuracy of Fus Device

To assess the in vivo targeting accuracy of FUS-BBBO performed using the FUS device disclosed herein, the following experiments were conducted. Mice were administered intravenously with a mixture of 1 mL/kg of Gadolinium (Dotarem, Guerbet, Aulnay sous Bois, France), 60 μL of 2% Evans Blue, and 10 μL/kg of Definity (Lantheus Medical Imaging, Billerica, Mass., USA) were subjected to FUS-BBBO using the disclosed device. FUS-BBB opening was measured by detecting gadolinium hyperenhancement within T1-weighted images obtained post-treatment, indicating the leakage of gadolinium associated with BBB disruption.

### Methods

### Animal Experimental Procedure

Adult female mice (IACUC protocol number: 21-0187, C57BL/6, 8 weeks old, female, Charles River Laboratory, Wilmington, Mass., USA) were used in this study. A total of 36 mice were randomly assigned to 9 groups to evaluate FUS-BBBO using FUS transducers with different frequencies (1.5, 3.0, 6.0 MHz) and different pressures (0.20, 0.40, 0.57 MPa) at each frequency. Prior to FUS sonication, animals were placed in the stereotaxic frame under isoflurane anesthesia (1.5-2% v/v isoflurane in oxygen). The fur on the mouse head was shaved using a hair removal cream while the skull and the scalp remained intact. Ultrasound gel was applied to the exposed skin above the skull and inside the housing of the small transducer. Mice were placed on a heating pad throughout the experiment. A catheter was placed in the mouse tail vein for intravenous injection.

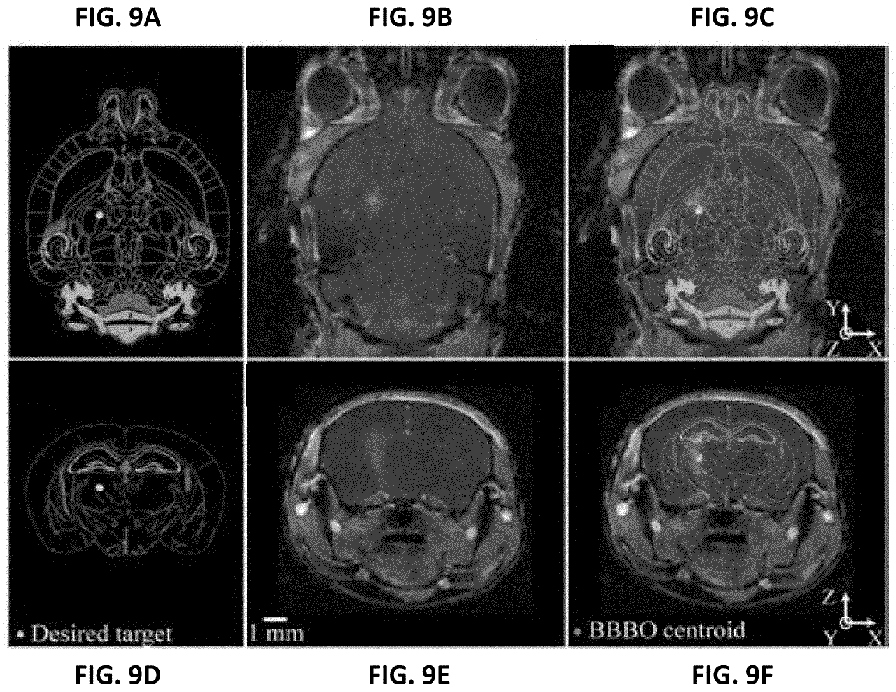

The desired brain target was selected to be a point in the left thalamus using the following coordinates: −1.94 mm in the anterior-posterior (AP) direction, −1.50 mm in the medial-lateral (ML) direction, and −3.30 mm in the dorsal-ventral (DV) direction relative to the bregma according to the mouse brain atlas. The desired target location is indicated by the yellow dot in the transverse view (FIG. 9A) and coronal view (FIG. 9D) of the mouse brain atlas. The FUS transducer was moved by the stereotactic frame to target the desired brain location. Then, a mixture of 1 mL/kg Gadolinium (Dotarem, Guerbet, Aulnay sous Bois, France), 60 μL of 2% Evans Blue, and 10 μL/kg of Definity (Lantheus Medical Imaging, Billerica, Mass., USA) was administered intravenously. It was followed by FUS sonication (pulse length 66 ms; pulse repetition frequency 5 Hz; duration 120 s) with different combinations of exposure frequency (1.5, 3.0, and 6.0 MHz) and pressure (0.20, 0.40, and 0.57 MPa). Approximately 5 minutes after FUS sonication ended, mice were imaged by a 4.7 T small animal MRI scanner (Agilent/Varian DirectDrive™ console, Agilent Technologies, Santa Clara, Calif., USA). A T1-weighted gradient-echo sequence was used for contrast-enhanced MRI (CE-MRI) using the following parameters: repetition time/echo time: 166 ms/6.4 ms; section thickness: 0.5 mm; in-plane resolution: 0.125×0.125 mm; matrix size: 256×256; number of signal averages: 2; flip angle: 60°). The BBB opening outcome was quantified based on hyperenhancement on the T1-weighted images, which indicated the leakage of gadolinium, as the intravenously injected gadolinium could not cross an intact BBB. Approximately 15 minutes after FUS sonication, mice were transcranial perfused with 0.01 M phosphate-buffered saline (PBS) followed by 4% paraformaldehyde. The mouse brains were then harvested and fixed in 4% paraformaldehyde for 24 hours before sectioning.

### Characterization of the Targeting Accuracy Using MRI

The targeting accuracy of the stereotactic-guided FUS device was quantified by the offset between the desired target location and the centroid of the BBBO detected by CE-MRI. Representative post-treatment CE-MRI images of the mouse brain are shown in FIGS. 2B and E. The hyperenhanced regions in the brain indicate the leakage of the MR contrast agent into the brain parenchyma, which represents the BBB opening area. The centroid of the BBB opening area was determined by finding the geometry center of hyperenhanced regions using Matlab. The offset between the coordinates used for targeting and the centroid of the BBB opening was calculated along three axes: medial-lateral (ML), anterior-posterior (AP), and dorsal-ventral (DV), corresponding to X-, Y-, and Z-axis in Cartesian coordinates, respectively.

### Quantification of Gadolinium Delivery Outcome

For each CE-MRI brain image, a region of interest was manually drawn to cover the non-treated right side of the brain. Three times the standard deviation above the mean pixel intensity within the non-treated region was calculated to represent the background intensity. Then, a region of interest was manually drawn to cover the FUS-treated left side of the brain. Pixels with intensities above the background intensity were identified for each CE-MRI image. Gadolinium delivery volume was calculated by the total number of the identified pixels in the whole brain.

### Results

### Gadolinium Delivery Outcome

MRI images in transverse and coronal views at different frequencies and pressures are shown in FIG. 12A. Gadolinium leakage was observed at 0.4 MPa and 0.57 MPa for all frequencies and 0.2 MPa for 1.5 MHz, whereas it was not detectable for 3.0 MHz and 6.0 MHz at 0.2 MPa. A summary of the mean Gadolinium delivery volume for all the FUS-treated groups is shown in FIG. 12B. The correlations between Gadolinium delivery volume with CI and MI are shown in FIGS. 12C and D. A strong linear correlation was found between the Gadolinium delivery volume and CI (R²=0.92). The correlation of MI with Gadolinium delivery volume was slightly lower (R²=0.83).

### Example 4—In Vivo Evaluation of Drug Delivery Using FUS-BBBO Mediated by Fus Device

To assess the efficacy of drug delivery via FUS-BBBO performed using the FUS device disclosed herein, the following experiments were conducted. Following the FUS-BBBO treatment as described in Example 3, the brains of the mice were harvested and harvested sections were fluorescence imaged using an LI-COR imaging system (Pearl Trilogy; 700 Channel laser source (Ex 785 nm/Em 820 nm); resolution 85 μm). Enhanced fluorescence signals associated with the delivery of Evans blue through the FUS-disrupted BBB were determined by comparing sections from FUS-BBBO and corresponding untreated brain halves.

### Methods

Evans Blue has been commonly used as a model drug for evaluating the FUS-BBBO drug delivery outcome. The harvested mouse brains were cut into 1-mm thick coronal sections using the mouse brain matrix (World Precision Instrument, Sarasota, Fla., USA). Fluorescence images of brain sections were taken using a LI-COR imaging system (Pearl Trilogy; 700 Channel laser source (Ex 785 nm/Em 820 nm); resolution 85 μm) and analyzed using Matlab (Mathworks, Natick, Mass., USA). Same to gadolinium delivery quantification, a region of interest was manually drawn to cover the non-treated right side of the brain for each brain section. Three times the standard deviation above the mean pixel intensity within the non-treated region was calculated to represent the background autofluorescence intensity. Then, a region of interest was manually drawn to cover the FUS-treated left side of the brain. Pixels with intensities above the background autofluorescence intensity were identified for each brain section. Evans blue delivery volume was calculated by the total number of pixels with enhanced fluorescence signal in all the brain sections. The mean fluorescence intensity of pixels with enhanced fluorescence signal in all the brain sections was calculated to represent the signal intensity of the delivered Evans blue.

### Results

### Evans Blue Delivery Outcome

Bright-field images of coronal sections of the mouse brains and corresponding fluorescence images are presented in FIG. 13A. Evans Blue extravasation was observed at 0.4 MPa and 0.57 MPa for all frequencies and 0.2 MPa for 1.5 MHz, whereas it was not detectable for 3.0 MHz and 6.0 MHz at 0.2 MPa. A summary of the mean Evans Blue delivery volume and signal intensity for all the FUS-treated groups is shown in FIG. 13B. The largest BBB opening volume was achieved at 1.5 MHz at a pressure of 0.57 MPa, yielding a 69±3.8 mm³ in Evans Blue delivery volume and 47±8.2 in signal intensity (FIG. 13B). The smallest BBB opening volume was achieved using 6 MHz with a pressure of 0.4 MPa, yielding 8.9±1.3 mm³ in delivery volume.

The correlation between Evans blue delivery volume and signal intensity with CI is shown in FIGS. 13A and C. The correlation between Evans blue delivery volume and signal intensity with MI is shown in FIGS. 13B and D. A strong linear correlation was found between the EB volume and CI (R²=0.91). The correlation of MI with EB volume was slightly lower (R²=0.84). The correlation between EB signal intensity and CI (R²=0.68) was comparable to that with MI (R²=0.71).

### Example 5—In Vivo Evaluation of Safety of FUS-BBBO Mediated by FUS Device

To assess the safety of FUS-BBBO performed using the FUS device disclosed herein, the following experiments were conducted. Following fluorescent microscope examination, a portion of the brain sections described in Example 3 containing the highest Evans blue fluorescence signal was subjected to histologic examination using hematoxylin and eosin (H&E) staining to detect microhemorrhage resulting from FUS-BBBO.

### Methods

### FUS-BBBO Safety Analysis

Histologic examination was performed for all mice using hematoxylin and eosin (H&E) staining. After fluorescence imaging, brain slices containing the highest Evans blue fluorescence signal were cryoprotected in 30% sucrose and embedded at −20° C. Brain slices were then sectioned into 5 μm coronal sections and stained with H&E. Bright-field images of stained sections were obtained using an all-in-one microscope (BZ-X810, Keyence, Osaka, Japan) coupled with 2× and 20× objectives.

### Statistical Analysis

Linear curve fitting was performed using OriginLab software (Origin, Mass., USA) for the following four groups: (a) Evans blue volume v.s. CI; (b) Evans blue volume v.s. MI; (c) Evans blue intensity v.s. CI; and (d) Evans blue intensity v.s. MI. The coefficient of determination (R²) was calculated. All data are presented in the format of mean±standard deviation. Statistical significance was evaluated by the unpair two-tail student t-test using GraphPad Prism (Version x, La Jolla, Calif., USA), and a p-value<0.05 was defined as statistically significant.

### Results

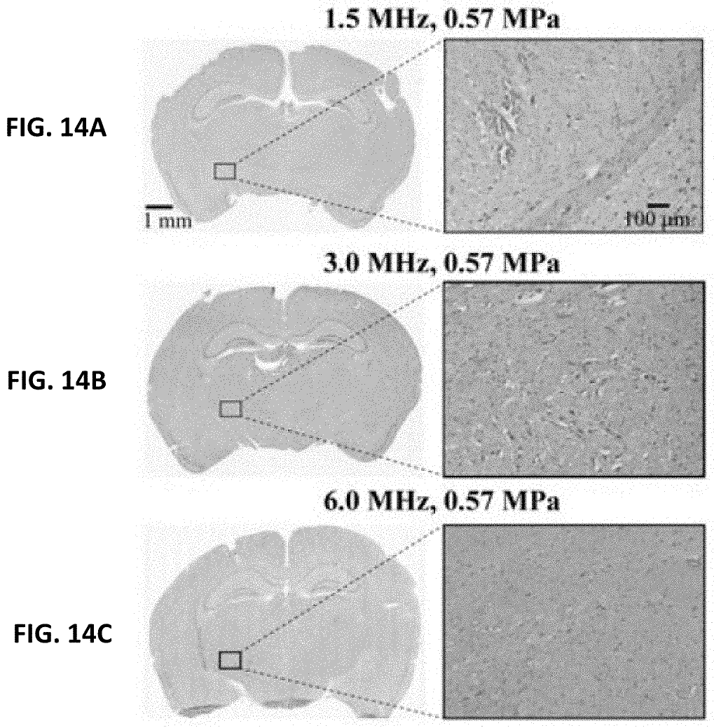

Of all groups tested, we only detected microhemorrhage in 2 out of 4 mice treated with FUS delivered at 1.5 MHz at 0.57 MPa. Representative slides with microhemorrhage observed are shown in FIG. 14A. No tissue damage was detected in other groups (FIGS. 14B and 14C).

What is claimed is:

1. A focused ultrasound (FUS) device for delivering FUS to a targeted tissue, comprising:
a. an adapter configured to an actuated mounting shaft of a stereotaxic system, the adapter comprising an adapter platform, the adapter platform defining:
i. an adapter platform defining a mounting bore at one end, the mounting bore configured to receive the actuated mounting shaft; and
ii. a transducer housing bore configured to receive a transducer housing;
b. the focused ultrasound (FUS) transducer; and
c. the transducer housing, each transducer housing comprising:
i. a transducer bore connecting a transducer receptacle and a coupling cone positioned on opposite faces of the transducer housing;
ii. the transducer receptacle configured to receive and house the focused ultrasound (FUS) transducer; and
iii. a coupling cone projecting downward from the transducer bore, the coupling cone configured to insert through the transducer housing bore.

2. The device of claim 1, wherein:
a. the adapter further comprises a first pair of magnets attached to the adapter within a corresponding pair of magnet insets formed within the adapter on either side of the transducer housing bore;
b. the transducer housing further comprises a second pair of magnets attached to the transducer housing within a corresponding second pair of magnet insets formed within the transducer housing on either side of the transducer bore;
wherein the first and second sets of magnets are configured to lock the transducer housing to the adapter when the coupling cone is inserted through the transducer housing bore.

3. The device of claim 1 wherein the coupling cone is further configured to contain an impedance-matched ultrasound coupling material, the impedance-matched ultrasound coupling material configured to provide a low-impedance acoustic path for delivery of FUS to the targeted tissue.

4. The device of claim 1, further comprising a pointer insert configured to insert into the transducer mounting bore and to provide a visual indication of the position and orientation of the FUS produced by the device, the pointer insert comprising:
a. a flanged insert configured to insert into the transducer mounting bore; and
b. a shaft extending downward and ending in a pointer tip, wherein the pointer tip is positioned at the focus point of the focused ultrasound produced by the device.

5. A focused ultrasound system, comprising:
a. an adapter configured to couple to an actuated mounting shaft of a stereotaxic system, the adapter comprising an adapter platform, the adapter platform defining:
i. an adapter platform defining a mounting bore at one end, the mounting bore configured to receive the actuated mounting shaft; and
ii. a transducer housing bore configured to receive a transducer housing;
b. the focused ultrasound (FUS) transducer; and
c. at least one transducer housing, each transducer housing comprising:
i. a transducer bore connecting a transducer receptacle and a coupling cone positioned on opposite faces of the transducer housing;
ii. the transducer receptacle configured to receive and house the focused ultrasound (FUS) transducer; and
iii. a coupling cone projecting downward from the transducer bore, the coupling cone configured to insert through the transducer housing bore.
d. a transducer driving system operatively coupled to the focused ultrasound (FUS) transducer, the transducer driving system comprising:
i. a function generator configured to control the operation of the FUS transducer to produce FUS; and
ii. a power amplifier configured to control the operation of the FUS transducer to produce FUS.

6. A stereotaxic-guided FUS-BBB system, comprising:
a. a focused ultrasound system, the focused ultrasound system comprising
i. an adapter configured to couple to an actuated mounting shaft of a stereotaxic system, the adapter comprising an adapter platform, the adapter platform defining:
1. an adapter platform defining a mounting bore at one end, the mounting bore configured to receive the actuated mounting shaft; and
2. a transducer housing bore configured to receive a transducer housing;
ii. a focused ultrasound (FUS) transducer; and
iii. at least one transducer housing, each transducer housing comprising:
1. a transducer bore connecting a transducer receptacle and a coupling cone positioned on opposite faces of the transducer housing;
2. the transducer receptacle configured to receive and house the focused ultrasound (FUS) transducer; and
3. a coupling cone projecting downward from the transducer bore, the coupling cone configured to insert through the transducer housing bore.
b. a stereotaxic system comprising an actuated mounting shaft, wherein the actuated mounting shaft is inserted into a mounting bore defined within the platform of the FUS device to couple the FUS device to the stereotaxic system.
